(19) United States
(12) Patent Application Publication
(10) Pub. No.: US 2026/0033723 A1
(43) Pub. Date: Feb. 5, 2026

(54) DETECTION DEVICE

(71) Applicant: Japan Display Inc., Tokyo (JP)

(72) Inventors: Kento HIMOTO, Tokyo (JP); Gen KOIDE, Tokyo (JP)

(21) Appl. No.: 19/352,968

(22) Filed: Oct. 8, 2025

Related U.S. Application Data
(63) Continuation of application No. PCT/JP2024/010245, filed on Mar. 15, 2024.

(30) Foreign Application Priority Data
Apr. 12, 2023 (JP) 2023-065203

(51) Int. Cl. A61B 5/00; G06V 40/13; G06V 40/145; G06V 40/40; H10K 39/32

(52) U.S. Cl. CPC A61B 5/0059; A61B 5/6826; G06V 40/1318; G06V 40/45; H10K 39/32; A61B 2562/0238; A61B 2562/185; G06V 40/145

(57) ABSTRACT

According to an aspect, a detection device includes: a substrate; a plurality of photodiodes in each of which a lower electrode, a lower buffer layer, an active layer, an upper buffer layer, and an upper electrode are stacked on the substrate in the order as listed; and a light-blocking layer provided in an area overlapping an edge region of the lower buffer layer, an edge region of the active layer, an edge region of the upper buffer layer, and an edge region of the lower electrode in plan view. The upper electrode covers the lower buffer layer, the active layer, the upper buffer layer, and the lower electrode. The lower buffer layer, the active layer, the upper buffer layer, and the lower electrode are arranged so as to be separated for each of the photodiodes.

### CROSS-REFERENCE TO RELATED APPLICATION

This application claims the benefit of priority from Japanese Patent Application No. 2023-065203 filed on Apr. 12, 2023 and International Patent Application No. PCT/JP2024/010245 filed on Mar. 15, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (refer to, for example, Japanese Patent Application Laid-open Publication No. 2009-032005). As such optical sensors, optical sensors that include a plurality of photodiodes (organic photodiodes (OPDs)) each using an organic semiconductor material as an active layer are known. The organic semiconductor material is disposed between lower and upper electrodes, and signal lines are electrically coupled to the lower electrodes of the photodiodes to output detection signals to a detection circuit.

Optical sensors that include such OPDs are required to have higher detection accuracy.

For the foregoing reasons, there is a need for a detection device capable of improving the detection accuracy.

### SUMMARY

According to an aspect, a detection device includes: a substrate; a plurality of photodiodes in each of which a lower electrode, a lower buffer layer, an active layer, an upper buffer layer, and an upper electrode are stacked on the substrate in the order as listed; and a light-blocking layer provided in an area overlapping an edge region of the lower buffer layer, an edge region of the active layer, an edge region of the upper buffer layer, and an edge region of the lower electrode in plan view. The upper electrode covers the lower buffer layer, the active layer, the upper buffer layer, and the lower electrode. The lower buffer layer, the active layer, the upper buffer layer, and the lower electrode are arranged so as to be separated for each of the photodiodes.

### BRIEF DESCRIPTION OF THE DRAWINGS

FIG. 1 is a schematic view illustrating an exemplary external appearance when a state of a finger accommodated inside a detection device according to a first embodiment is viewed from a lateral side of a housing;

FIG. 2 is a schematic sectional view taken along section II-II′ illustrated in FIG. 1;

FIG. 3 is a development view illustrating an exemplary development of optical sensors of the detection device illustrated in FIG. 1;

FIG. 4 is a configuration diagram illustrating an exemplary configuration of a first optical sensor and a second optical sensor illustrated in FIG. 3;

FIG. 5 is a schematic sectional view illustrating an exemplary multilayer configuration of the optical sensor taken along section V-V′ illustrated in FIG. 4;

FIG. 6 is a timing waveform diagram illustrating photoresponse characteristics of a detection device according to a comparative example;

FIG. 7 is a timing waveform diagram illustrating the photoresponse characteristics of the detection device;

FIG. 8 is a schematic sectional view illustrating an exemplary multilayer configuration of the optical sensor of a detection device according to a first modification of the first embodiment;

FIG. 9 is a development view illustrating an exemplary development of the optical sensor of a detection device according to a second modification of the first embodiment;

FIG. 10 is a configuration diagram illustrating an exemplary configuration of the optical sensor illustrated in FIG. 9;

FIG. 11 is a plan view schematically illustrating a detection device according to a second embodiment;

FIG. 12 is a block diagram illustrating an exemplary configuration of the detection device according to the second embodiment;

FIG. 13 is a circuit diagram illustrating the detection device according to the second embodiment;

FIG. 14 is a magnified schematic configuration view of a sensor according to the second embodiment;

FIG. 15 is a plan view illustrating a light-blocking layer according to the second embodiment;

FIG. 16 is a sectional view taken along XVI-XVI′ of FIG. 15;

FIG. 17 is a sectional view taken along XVII-XVII′ of FIG. 15;

FIG. 18 is a plan view illustrating a light-blocking layer according to a first modification of the second embodiment;

FIG. 19 is a plan view illustrating the light-blocking layer according to a second modification of the second embodiment;

FIG. 20 is a plan view illustrating a light-blocking layer according to a third modification of the second embodiment;

FIG. 21 is a sectional view taken along XXI-XXI′ of FIG. 20; and

FIG. 22 is a sectional view taken along XXII-XXII′ of FIG. 20.

### DETAILED DESCRIPTION

The following describes modes (embodiments) for carrying out the disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### First Embodiment

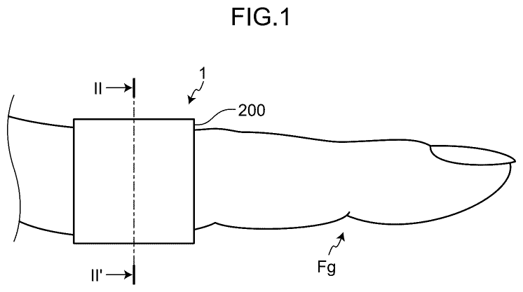

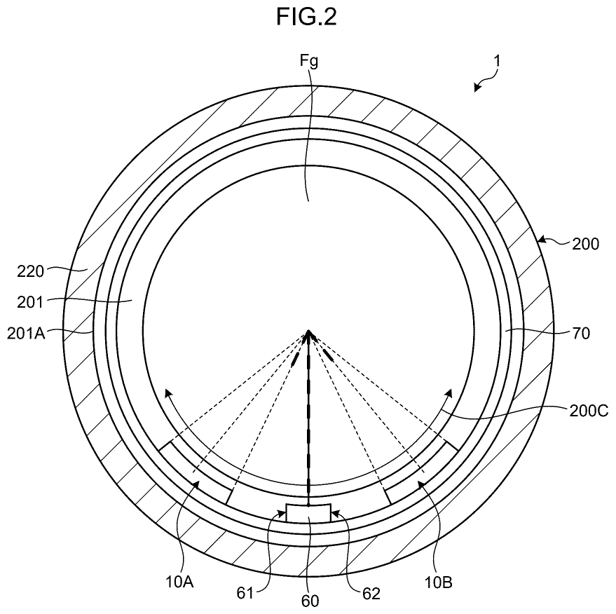

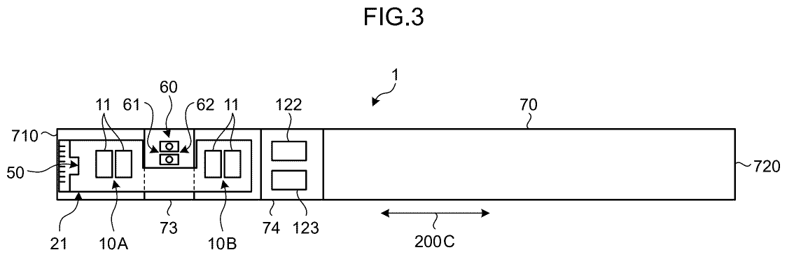

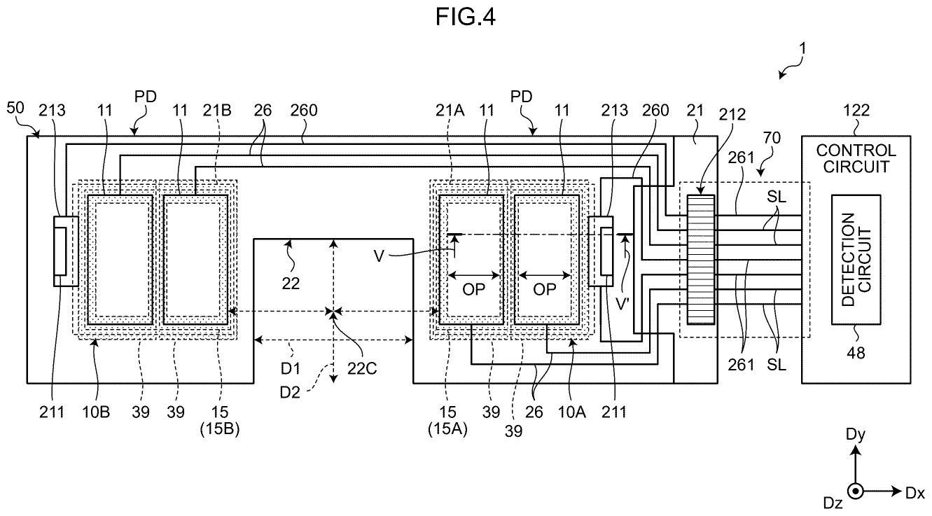

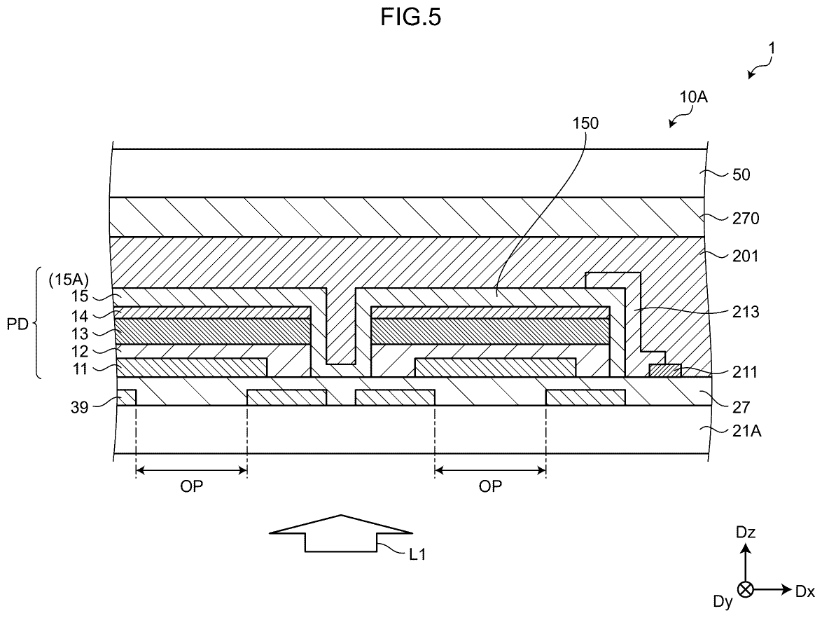

FIG. 1 is a schematic view illustrating an exemplary external appearance when a state of a finger accommodated inside a detection device according to a first embodiment is viewed from a lateral side of a housing. FIG. 2 is a schematic sectional view taken along section II-II′ illustrated in FIG. 1. FIG. 3 is a development view illustrating an exemplary development of optical sensors of the detection device illustrated in FIG. 1. FIG. 4 is a configuration diagram illustrating an exemplary configuration of a first optical sensor and a second optical sensor illustrated in FIG. 3. FIG. 5 is a schematic sectional view illustrating an exemplary multilayer configuration of the optical sensor taken along section V-V′ illustrated in FIG. 4.

A detection device 1 illustrated in FIG. 1 is a finger ring-shaped device that can be worn on and removed from a human body and is worn on a finger of the human body that is an object to be detected Fg. Examples of the finger, which is one example of the object to be detected Fg, include a thumb, an index finger, a middle finger, a ring finger, and a little finger. The human body is a person to be authenticated whose identity is to be verified by the detection device 1. The detection device 1 can detect biometric information on a living body from the finger wearing the detection device 1. The measurement target is the living body or a part of the living body, and is an object to be measured. The detection device 1 is formed as a finger ring or a wristband so as to be easily carried by a user. In the following description, the detection device 1 is assumed to be used as a finger ring.

As illustrated in FIG. 2, the detection device 1 includes a housing 200, a light source 60, a first optical sensor 10A, and a second optical sensor 10B. The detection device 1 is a device that includes a battery (not illustrated) in the housing 200 and is operated by power from the battery.

The housing 200 is formed into a ring shape (annular shape) that can be worn on the object to be detected Fg, and is a wearable member to be worn on the living body. In the example illustrated in FIG. 2, the housing 200 includes a sealing film 201 and an exterior member 220. The sealing film 201 is integrated with the exterior member 220 to form the housing 200 into the ring shape. The sealing film 201 accommodates therein the light source 60, the first optical sensor 10A, the second optical sensor 10B, and so forth. The sealing film 201 is formed into a ring shape using a housing material such as a light-transmitting synthetic resin or silicon. The exterior member 220 has a surface of the housing 200 that covers an outer peripheral surface 201A of the sealing film 201. The exterior member 220 is formed into a ring shape using a member of, for example, a metal, a non-light-transmitting synthetic resin, or the like. The housing 200 accommodates, in the sealing film 201, a flexible printed circuit board 70 on which the light source 60, the first optical sensor 10A, the second optical sensor 10B, and so forth are mounted. The flexible printed circuit board 70 is accommodated in the housing 200, for example, by forming the housing 200 by filling the periphery of the flexible printed circuit board 70 formed into a ring shape with a filling member in a mold.

As illustrated in FIG. 3, the number of elements of the optical sensors arranged in the detection device 1 illustrated in FIG. 1 is four. The flexible printed circuit board 70 is formed into a deformable band shape, and is formed into a ring shape by coupling one end 710 to the other end 720. The flexible printed circuit board 70 has a first mounting area 73 and a second mounting area 74. The first mounting area 73 is an area where the light source 60 and so forth are mounted. The second mounting area 74 is an area where a control circuit 122, a power supply circuit 123, and so forth are mounted. A first substrate 21 is mounted on the flexible printed circuit board 70 so as to straddle the vicinity of the light source 60 in the first mounting area 73. A second substrate 50 is provided on the first substrate 21. The flexible printed circuit board 70 electrically couples the light source 60, the first optical sensor 10A, the second optical sensor 10B, and so forth to the control circuit 122.

In the present embodiment, the first and the second optical sensors 10A and 10B are provided so as to interpose the light source 60 therebetween in a circumferential direction 200C. That is, in the detection device 1, the first optical sensor 10A, the light source 60, and the second optical sensor 10B are arranged in this order in the circumferential direction 200C. The first and the second optical sensors 10A and 10B are arranged so as to interpose the light source 60 therebetween in the circumferential direction 200C. Thereby, light emitted by the light source 60 can be detected over a wide area of the housing 200.

The first substrate 21 is an insulating substrate, and is formed, for example, of polyethylene terephthalate (PET) or the like that is a film-like synthetic resin and into a band shape. The first substrate 21 is a deformable substrate on which the first and the second optical sensors 10A and 10B are mounted. The first substrate 21 can be bent in a third direction Dz. When the sensor substrate 21 is mounted on the flexible printed circuit board 70, the first and the second optical sensors 10A and 10B are positioned on opposite sides of the light source 60 in the circumferential direction 200C of the housing 200. The first substrate 21 has a first area 21A where the first optical sensor 10A is mounted, and a second area 21B where the second optical sensor 10B is mounted. The first substrate 21 is formed as one substrate having the first area 21A and the second area 21B.

As with the first substrate 21, the second substrate 50 is an insulating substrate and is formed into a band shape composed, for example, of polyethylene terephthalate (PET) that is a film-like synthetic resin. The second substrate 50 covers the sealing film 201 and is a deformable substrate. The second substrate 50 can be bent in the third direction Dz.

In the present embodiment, as illustrated in FIG. 2, the flexible printed circuit board 70 is accommodated in the housing 200 such that a surface provided with the first optical sensor 10A, the second optical sensor 10B, and the light source 60 faces an inner peripheral surface 200B of the housing 200. When the flexible printed circuit board 70 has a light-transmitting property, the first optical sensor 10A, the second optical sensor 10B, and the light source 60 may be mounted on the back surface opposite the front surface. In this case, the light source 60 only needs to be disposed such that light is emitted toward the flexible printed circuit board 70 and light transmitted through the flexible printed circuit board 70 is emitted toward outside the housing 200.

As illustrated in FIG. 2, the light source 60 is provided in the sealing film 201 of the housing 200 and is configured to be capable of emitting light toward the object to be detected Fg wearing the ring-shaped housing 200. For example, inorganic light-emitting diodes (LEDs) or organic electroluminescent (EL) diodes (organic light-emitting diodes (OLEDs)) are used as the light source 60. The light source 60 emits light having predetermined wavelengths. In the present embodiment, the light source 60 includes a plurality of light sources so as to be capable of emitting near-infrared light, red light, and green light.

The light emitted from the light source 60 is reflected by a surface of the object to be detected Fg, and enters the first and the second optical sensors 10A and 10B. Thereby, the detection device 1 can detect a fingerprint by detecting a shape of asperities on the surface of the object to be detected Fg or the like. Alternatively, the light emitted from the light source 60 may be reflected in the object to be detected Fg, or transmitted through the object to be detected Fg and enter the first and the second optical sensors 10A and 10B. As a result, the detection device 1 can detect information on a living body in the finger or the like. Examples of the information on the living body include, but are not limited to, pulse waves, pulsation, and a vascular image in the finger or a palm. That is, the detection device 1 may be configured as a fingerprint detection device that detects the fingerprint or a vein detection device that detects a pattern of blood vessels such as veins.

Each of the first and the second optical sensors 10A and 10B detects the light emitted by the light source 60 and reflected by the object to be detected Fg, light directly incident on the optical sensor, and other light. The first and the second optical sensors 10A and 10B are each an organic photodiode (OPD). The first optical sensor 10A is provided in the housing 200 so as to be adjacent to one end 610 of the light source 60 in the circumferential direction 200C of the housing 200. The second optical sensor 10B is provided in the housing 200 so as to be adjacent to another end 620 of the light source 60 in the circumferential direction 200C of the housing 200.

As illustrated in FIG. 3, the first and the second optical sensors 10A and 10B each include a photodiode PD (refer to FIG. 4) that is an organic photodiode. Each of the first and the second optical sensors 10A and 10B has a configuration with two lower electrodes 11 arranged along the circumferential direction 200C. The first and the second optical sensors 10A and 10B are mounted on one first substrate 21 and are electrically coupled to the flexible printed circuit board 70 via the first substrate 21. The first substrate 21 has a notch 22 (refer to FIG. 4) between the first and the second optical sensors 10A and 10B in the circumferential direction 200C of the housing 200.

In the following description, a first direction Dx is one direction in a plane parallel to the first substrate 21 and is the same direction as the circumferential direction 200C. A second direction Dy is one direction in the plane parallel to the first substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the first substrate 21. The term “plan view” refers to a positional relation when viewed along a direction orthogonal to the first substrate 21.

As illustrated in FIG. 4, the first optical sensor 10A has a configuration in which the two lower electrodes 11 arranged in the first direction Dx and one upper electrode 15A are stacked together. The second optical sensor 10B has a configuration in which the two lower electrodes 11 arranged in the first direction Dx and one upper electrode 15B are stacked together. An upper electrode 15 includes the upper electrode 15A of the first optical sensor 10A and the upper electrode 15B of the second optical sensor 10B. Each of the upper electrode 15A and the upper electrode 15B covers the two lower electrodes 11 in plan view.

The first substrate 21 includes a power supply electrode 211 that extends along the second direction Dy. The power supply electrode 211 is electrically coupled to a coupling part 212 (terminal) of the first substrate 21, and is supplied with a sensor power supply signal (sensor power supply voltage) from the power supply circuit 123 (refer to FIG. 3) via the coupling part 212. The upper electrode 15 is electrically coupled to the power supply electrode 211 by a conductor 213. The conductor 213 is provided on the first substrate 21 so as to extend overlapping both the upper electrode 15 and the power supply electrode 211, and is formed of a conductive material. With this configuration, the upper electrode 15 is supplied with the sensor power supply signal from the power supply circuit 123 via the power supply electrode 211.

A plurality of first wiring lines 26 of the first substrate 21 are coupled to a detection circuit 48 included in the control circuit 122 via a plurality of signal lines SL of the flexible printed circuit board 70. The detection circuit 48 is electrically coupled to the lower electrodes 11 of the first and the second optical sensors 10A and 10B via the signal lines SL. The detection circuit 48 may be formed as a circuit separate from the control circuit 122.

The first wiring lines 26 are formed, for example, of metal wiring, and is formed of a material having better conductivity than the lower electrodes 11 of the photodiode PD. The first wiring lines 26 are formed of a light-transmitting conductive material such as indium tin oxide (ITO). The first wiring lines 26 are provided in a layer between the first substrate 21 and the photodiode PD in the third direction Dz. The first wiring lines 26 are electrically coupled to the lower electrodes 11 and the coupling part 212 of the first substrate 21. The first wiring lines 26 may be formed, for example, in the same layer as the lower electrodes 11, or formed of a metal.

Second wiring lines 260 are electrically coupled to the power supply electrode 211 by the conductor 213. The second wiring lines 260 are formed, for example, of metal wiring, and is formed of a conductive material. The second wiring lines 260 are formed of a material having better conductivity than the upper electrode 15. The second wiring lines 260 are provided in a layer between the first substrate 21 and the photodiode PD in the third direction Dz. The second wiring lines 260 are electrically coupled to the upper electrode 15 and the coupling part 212. The second wiring lines 260 may be formed, for example, in the same layer as the upper electrode 15, or formed of a metal. The second wiring lines 260 may be a shield layer.

The control circuit 122 is a circuit that controls detection operations by supplying control signals to the photodiodes PD. Each of the photodiodes PD outputs an electrical signal in response to the light emitted thereto as a detection signal Vdet to the detection circuit 48. The second wiring lines 260 are coupled to the control circuit 122 via wiring lines 261 that supply a power supply voltage to the second wiring lines 260. In the present embodiment, the detection signals Vdet of the photodiodes PD are sequentially output to the detection circuit 48 in a time-division manner. In other words, the signal lines SL are sequentially electrically coupled to the detection circuit 48 in a time-division manner. Thereby, the detection device 1 detects information on the object to be detected Fg based on the detection signals Vdet from the photodiodes PD.

As illustrated in FIG. 5, the first optical sensor 10A includes the first substrate 21 (first area 21A), the photodiodes PD, and the second substrate 50 that faces the first substrate 21. In the present embodiment, the first optical sensor 10A further includes a first insulating layer 27 and a second insulating layer 270.

The first insulating layer 27 is provided on the upper side of the first substrate 21. The first insulating layer 27 is located between the first substrate 21 and the photodiode PD. The second insulating layer 270 is provided on the upper side of the photodiode PD. The second insulating layer 270 is located between the second substrate 50 and the photodiode PD. The first insulating layer 27 and the second insulating layer 270 may be inorganic insulating films or organic insulating films.

The photodiode PD is provided on the upper side of the first insulating layer 27. The photodiode PD includes the lower electrodes 11, a lower buffer layer 12, an active layer 13, an upper buffer layer 14, and the upper electrode 15 (15A). In the photodiode PD, the lower electrodes 11, the lower buffer layer 12 (hole transport layer), the active layer 13, the upper buffer layer 14 (electron transport layer), and the upper electrode 15 are stacked on the upper side of a light-blocking layer 39 in this order in the third direction Dz orthogonal to the first substrate 21. The lower buffer layer 12, the active layer 13, the upper buffer layer 14, and the lower electrodes 11 are provided so as to be separated for each of the photodiodes PD.

Each of the lower electrodes 11 is an anode electrode of the photodiode PD and is formed of a light-transmitting conductive material such as indium tin oxide (ITO), for example.

The active layer 13 changes in characteristics (such as voltage-current characteristics and resistance value) depending on light emitted thereto. An organic material is used as a material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using a material obtained by combining any of the above-listed low-molecular-weight organic materials with a high-molecular-weight organic material. As the high-molecular-weight organic material, for example, poly(3-hexylthiophene) (P₃HT) and F8-alt-benzothiadiazole (F8BT) can be used. The active layer 13 can be a film made of a mixture of P3HT and PCBM, or a film made of a mixture of F8BT and PDI.

The lower buffer layer 12 is a hole transport layer. The upper buffer layer 14 is an electron transport layer. The lower buffer layer 12 and the upper buffer layer 14 are provided to facilitate holes and electrons generated in the active layer 13 to reach the lower electrodes 11 or the upper electrode 15. The lower buffer layer 12 (hole transport layer) is in direct contact with the tops of the lower electrodes 11 and is also provided in an area between the adjacent lower electrodes 11. The active layer 13 is in direct contact with the top of the lower buffer layer 12. The material of the hole transport layer is a metal oxide layer. Tungsten oxide (WO₃), molybdenum oxide, or the like is used as the metal oxide layer.

The upper buffer layer 14 (electron transport layer) is in direct contact with the top of the active layer 13, and the upper electrode 15 is in direct contact with the top of the upper buffer layer 14. Polyethylenimine ethoxylated (PEIE) is used as a material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film, and may be formed as a multilayered film that includes an electron blocking layer and a hole blocking layer.

The upper electrode 15 is provided on the upper buffer layer 14. The upper electrode 15 is a cathode electrode of the photodiode PD, and is continuously formed over the entire first and second optical sensors 10A and 10B. In other words, the upper electrode 15 is continuously provided on the photodiodes PD. The upper electrode 15 faces the lower electrodes 11 with the lower buffer layer 12, the active layer 13, and the upper buffer layer 14 interposed therebetween. The upper electrode 15 is formed, for example, of a light-transmitting conductive material such as ITO or indium zinc oxide (IZO). The upper electrode 15 may be a multilayered film of a plurality of light-transmitting conductive materials. A portion of an edge region of an upper surface 150 of the upper electrode 15 is electrically coupled to the conductor 213. In the first optical sensor 10A, the photodiode PD is well sealed by providing the sealing film 201 on the upper electrode 15 and so forth. The upper electrode 15 is provided on the upper buffer layers 14 so as to extend across the adjacent photodiodes PD.

As illustrated in FIGS. 4 and 5, the light-blocking layer 39 is provided between the first substrate 21 and the first insulating layer 27 in the third direction Dz, in an area overlapping an outer edge region of the upper electrode 15 for each of the photodiodes PD. The light-blocking layer 39 is provided in an area overlapping an edge region of the lower buffer layer 12, an edge region of the active layer 13, an edge region of the upper buffer layer 14, and edge regions of the lower electrodes 11 in plan view. The light-blocking layer 39 is formed of a non-light-transmitting material. The light-blocking layer 39 is provided so as to cover the edge region of the upper electrode 15.

An opening OP is formed in an area of the light-blocking layer 39 overlapping the lower electrode 11.

The second optical sensor 10B includes two adjacent lower electrodes 11 of the second optical sensor 10B in the second area 21B of the first substrate 21 different from the area for the lower electrodes 11 of the first optical sensor 10A. The second optical sensor 10B includes the first substrate 21 (second area 21B), the photodiode PD, the first insulating layer 27, the second substrate 50 that faces the first substrate 21, and the second insulating layer 270. The photodiode PD of the second optical sensor 10B has the same configuration as that of the photodiode PD of the first optical sensor 10A. That is, the photodiode PD of the second optical sensor 10B includes the lower electrodes 11, the lower buffer layer 12, the active layer 13, the upper buffer layer 14, and the upper electrode 15 (15B). In the present embodiment, the photodiodes PD of the first and the second optical sensors 10A and 10B are organic photodiodes.

As illustrated in FIG. 4, the first substrate 21 has the first area 21A of the first optical sensor 10A and the second area 21B of the second optical sensor 10B, and is integrally formed into one common substrate. In the first substrate 21, the notch 22 is formed between the first area 21A of the first optical sensor 10A and the second area 21B of the second optical sensor 10B in the first direction Dx. The first substrate 21 has the notch 22 between the first and the second optical sensors 10A and 10B.

The notch 22 is formed to have a length D1 longer than the length of the light source 60 in the first direction Dx. The notch 22 is formed to have a length D2 longer than the length of the light source 60 and shorter than the length (width) of the first substrate 21 in the second direction Dy. The notch 22 is formed such that the distance between a center 22C and one side of the lower electrode 11 of the first optical sensor 10A is equal to the distance between the center 22C and one side of the lower electrode 11 of the second optical sensor 10B in the first direction Dx. In the first embodiment, the notch 22 is formed into a substantially rectangular shape in plan view, but may have a semicircular, triangular, polygonal, or other shape, for example.

If the detection device 1 is a bottom-illuminated optical sensor, the lower buffer layer 12 is an electron transport layer and the upper buffer layer 14 is a hole transport layer. If the detection device 1 is a top-illuminated optical sensor, the lower buffer layer 12 is a hole transport layer and the upper buffer layer 14 is an electron transport layer. The active layer 13 is in direct contact with the top of the lower buffer layer 12. The material of the hole transport layer is a metal oxide layer. Tungsten oxide (WO₃), molybdenum oxide, or the like is used as the metal oxide layer.

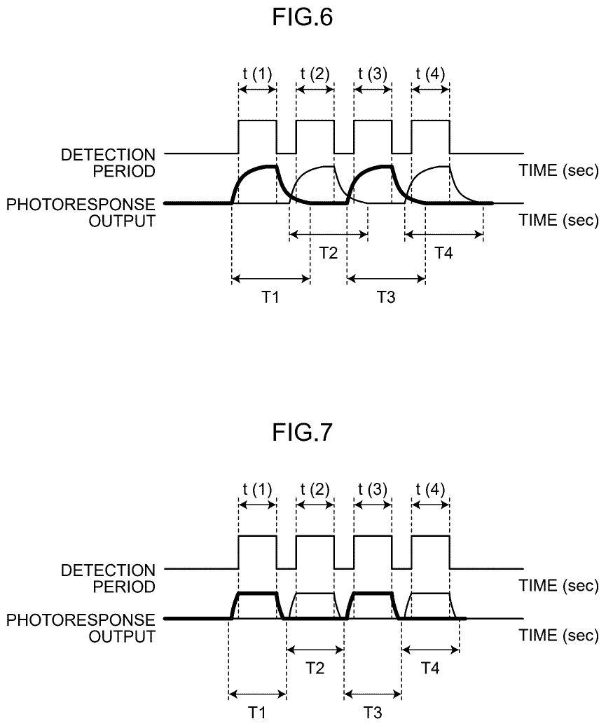

FIG. 6 is a timing waveform diagram illustrating photoresponse characteristics of a detection device according to a comparative example. FIG. 7 is a timing waveform diagram illustrating the photoresponse characteristics of the detection device. The light-blocking layer 39 is not provided in a structure of the comparative example.

As illustrated in FIGS. 6 and 7, a detection operation using first light is performed during periods t(1) and t(3), and the detection operation using second light is performed during periods t(2) and t(4). Hereinafter, the periods t(1) and t(3) during which the detection operation using the first light is performed are also referred to as “first light detection periods” and the periods t(2) and t(4) during which the detection operation using the second light is performed are also referred to as “second light detection periods”.

Examples of the first light include near-infrared and green light, and examples of the second light include red light. When the detection operation using the first light is performed, photoresponsive components T1 and T3 of carriers (holes and electrons) are generated by the active layer 13. When the detection operation using the second light is performed, photoresponsive components T2 and T4 of the carriers (holes and electrons) are generated by the active layer 13.

If the light-blocking layer 39 is not provided in the edge region of the lower electrode 11 of the detection device according to the comparative example, after the first light or the second light is detected, a delayed response component of the first light or the second light remains in the next detection period, and a mixture of the first light and the second light is detected as illustrated in FIG. 6. Thus, if the light-blocking layer 39 is not provided, the carriers (holes and electrons) generated in the active layer 13 in an area overlapping the edge region of the lower electrode 11 may have a delayed photoresponse until reaching the lower electrode 11 compared with the carriers (holes and electrons) generated in the active layer 13 in an area not overlapping the edge region of the lower electrode 11.

In contrast thereto, in the first embodiment, the light-blocking layer 39 is provided in the edge region of the lower electrode 11 of the detection device 1. Thus, the edge region of the lower electrode 11 is shielded from light. Consequently, as illustrated in FIG. 7, peaks of the photoresponse output are lower than peaks of the photoresponse output illustrated in FIG. 6. As a result, after the first light or the second light is detected, the delayed response component of the first light or the second light does not remain and does not affect the optical response component of the first light or the second light in the next detection period.

The detection device 1 of the present embodiment is configured as the bottom-illuminated optical sensor. That is, light L1 emitted from the light source 60 (refer to FIG. 2) and transmitted through or reflected by the object to be detected Fg is transmitted through the first substrate 21 and is irradiated onto the lower electrode 11 of the photodiode PD. The light L1 is transmitted through the opening OP in the light-blocking layer 39 and is irradiated onto the active layer 13 of the photodiode PD. The carriers (holes and electrons) generated in the active layer 13 reach the lower electrode 11 and the upper electrode 15 through the lower buffer layer 12 and the upper buffer layer 14, respectively.

In the detection device 1, the light L1 is blocked in an area overlapping the light-blocking layer 39 and does not reach the active layer 13 located in the area overlapping the light-blocking layer 39. As a result, the generation of the carriers (holes and electrons) is reduced in a portion of the active layer 13 overlapping the light-blocking layer 39 (portion overlapping the outer edge region of the lower electrode 11). Therefore, occurrence of a delay in arrival time of the carriers (holes and electrons) generated in the active layer 13 can be reduced between a portion of the photodiode PD overlapping the outer edge region of the lower electrode 11 and a portion of the photodiode PD not overlapping the outer edge region of the lower electrode 11. As a result, the detection device 1 including the OPD can improve the detection accuracy.

The exemplary configuration of the detection device 1 according to the present embodiment has been described above. The configuration described above using FIGS. 1 to 5 is merely an example, and the configuration of the detection device 1 according to the present embodiment is not limited to the example. The configuration of the detection device 1 according to the present embodiment can be flexibly modified depending on specifications or operations.

### First Modification of First Embodiment

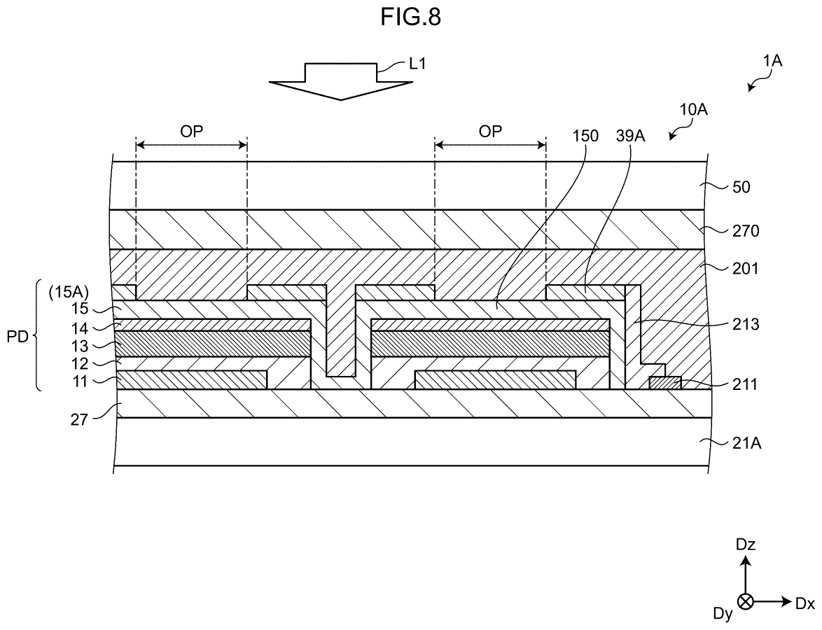

FIG. 8 is a schematic sectional view illustrating an exemplary multilayer configuration of the optical sensor of a detection device according to a first modification of the first embodiment. In the following description, the same components as those described in the embodiment above are denoted by the same reference numerals, and the description thereof will not be repeated.

As illustrated in FIG. 8, in a detection device 1A according to the first modification of the first embodiment, a light-blocking layer 39A is provided in the outer edge region of the upper electrode 15 for each of the photodiodes PD.

The detection device 1A is configured as a top-illuminated optical sensor. That is, the light L1 emitted from the light source 60 (refer to FIG. 2) and transmitted through or reflected by the object to be detected Fg is transmitted through the first insulating layer 27 and is irradiated onto the upper electrode 15 of the photodiode PD. The light L1 is transmitted through the opening OP in the light-blocking layer 39A and is irradiated onto the active layer 13 of the photodiode PD. The carriers (holes and electrons) generated in the active layer 13 reach the lower electrode 11 and the upper electrode 15 through the lower buffer layer 12 and the upper buffer layer 14, respectively.

Also, in the detection device 1A, the light L1 is blocked in an area overlapping the light-blocking layer 39A, and does not reach a portion of the active layer 13 overlapping the light-blocking layer 39A. As a result, the generation of the carriers (holes and electrons) is reduced in a portion of the active layer 13 overlapping the light-blocking layer 39A (portion overlapping the area of the outer edge region of the lower electrode 11). As a result, the detection device 1A including the OPD can improve the detection accuracy.

### Second Modification of First Embodiment

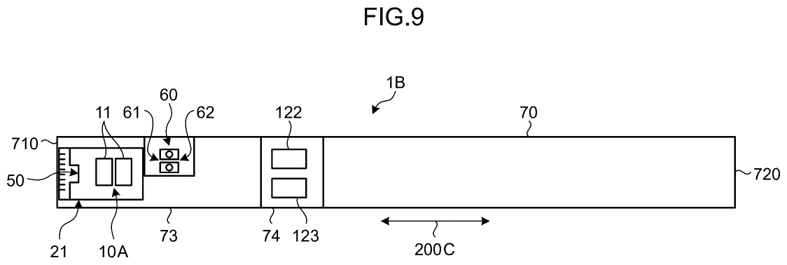

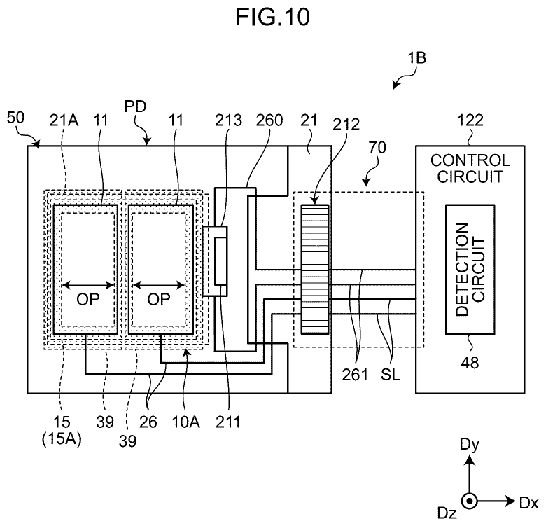

FIG. 9 is a development view illustrating an exemplary development of the optical sensor of a detection device according to a second modification of the first embodiment. FIG. 10 is a configuration diagram illustrating an exemplary configuration of the optical sensor illustrated in FIG. 9. In the following description, the same components as those described in the embodiment above are denoted by the same reference numerals, and the description thereof will not be repeated.

As illustrated in FIG. 9, the number of elements of the optical sensor arranged in a detection device 1B is two. The flexible printed circuit board 70 electrically couples the light source 60, the first optical sensor 10A, and so forth to the control circuit 122. In the detection device 1B, the first optical sensor 10A and the light source 60 are arranged in this order in the circumferential direction 200C. The first optical sensor 10A is located near one end side of the light source 60 in the circumferential direction 200C. Thereby, the light emitted by the light source 60 can be detected over a wide area of the housing 200.

When the first substrate 21 is mounted on the flexible printed circuit board 70, the first optical sensor 10A is positioned near the one end side of the light source 60 in the circumferential direction 200C of the housing 200. The first substrate 21 has the first area 21A where the first optical sensor 10A is mounted. The first substrate 21 is formed as one substrate having the first area 21A.

As illustrated in FIG. 10, the first optical sensor 10A has a configuration in which two lower electrodes 11 arranged in the first direction Dx and one upper electrode 15A are stacked together. The upper electrode 15 includes the upper electrode 15A of the first optical sensor 10A. The upper electrode 15A covers the two lower electrodes 11 in plan view.

### Second Embodiment

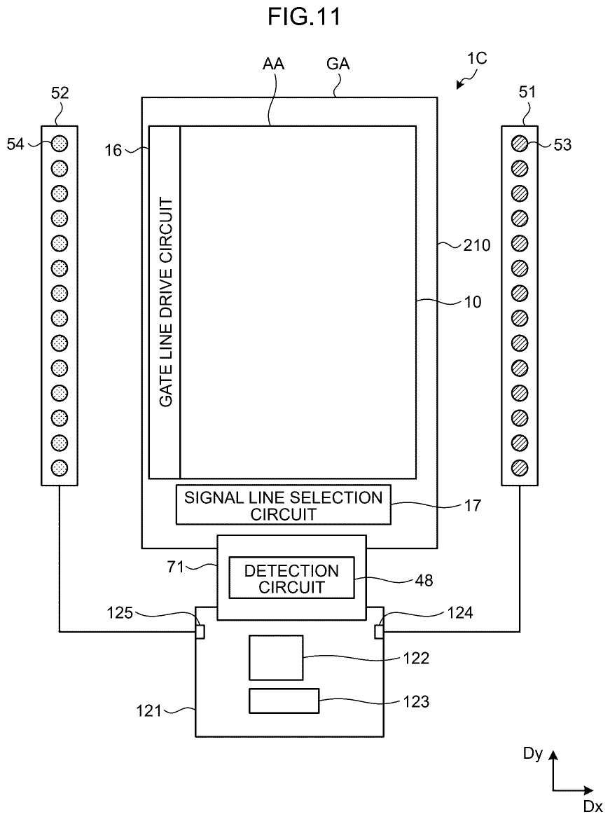

FIG. 11 is a plan view schematically illustrating a detection device according to a second embodiment. In the following description, the same components as those described in the embodiment above are denoted by the same reference numerals, and the description thereof will not be repeated.

As illustrated in FIG. 11, a detection device 1C includes a sensor base member 210 (substrate), a sensor 10, a gate line drive circuit 16, a signal line selection circuit 17, the detection circuit 48, the control circuit 122, the power supply circuit 123, a first light source base member 51, a second light source base member 52, and light sources 53 and 54. The first light source base member 51 is provided with a plurality of the light sources 53. The second light source base member 52 is provided with a plurality of the light sources 54.

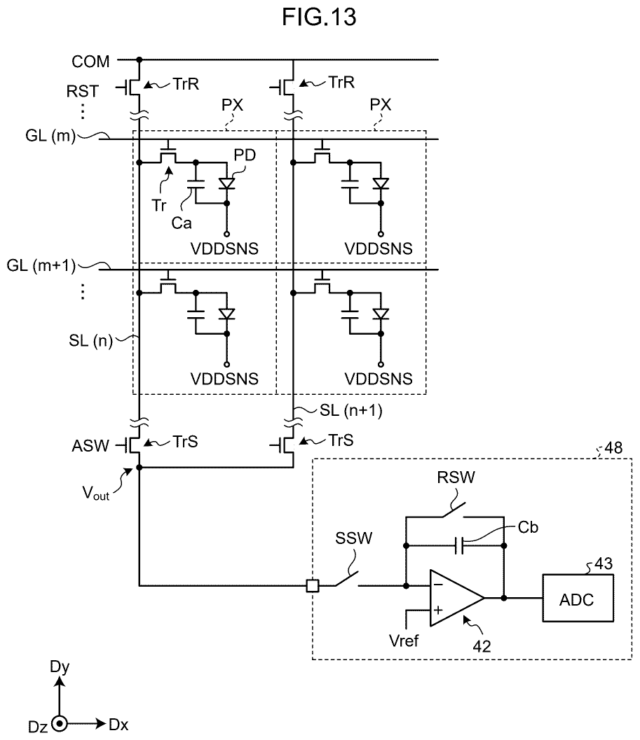

The sensor base member 210 is electrically coupled to a control substrate 121 via a wiring substrate 71. The wiring substrate 71 is, for example, a flexible printed circuit board or a rigid circuit board. The wiring substrate 71 is provided with the detection circuit 48. The control substrate 121 is provided with the control circuit 122 and the power supply circuit 123. The control circuit 122 is a field-programmable gate array (FPGA), for example. The control circuit 122 supplies control signals to the sensor 10, the gate line drive circuit 16, and the signal line selection circuit 17 to control detection operations of the sensor 10. The control circuit 122 supplies control signals to the light sources 53 and 54 to control lighting and non-lighting of the light sources 53 and 54. The power supply circuit 123 supplies voltage signals such as a sensor power supply signal (sensor power supply voltage) VDDSNS (refer to FIG. 13) to the sensor 10, the gate line drive circuit 16, and the signal line selection circuit 17. The power supply circuit 123 supplies a power supply voltage to the light sources 53 and 54.

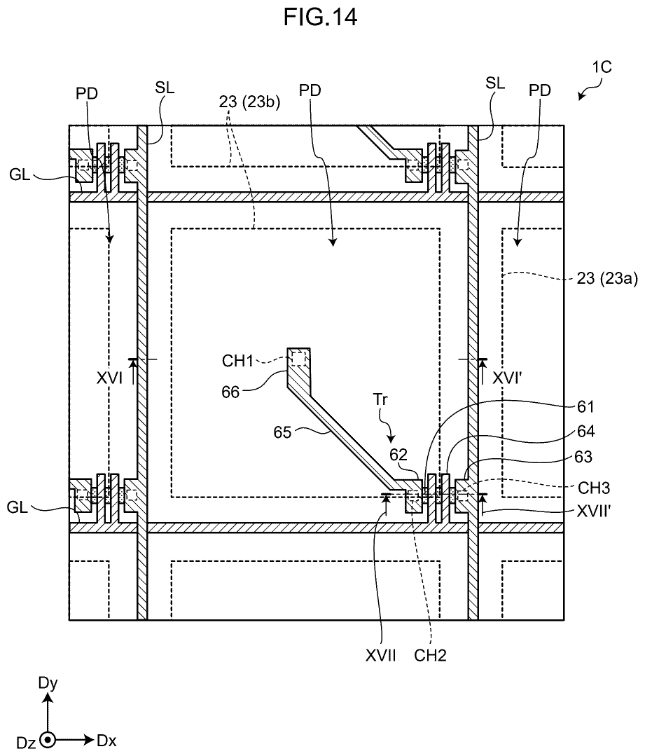

The sensor base member 210 has a detection area AA and a peripheral area GA. The detection area AA is an area provided with the photodiodes PD (refer to FIG. 14) included in the sensor 10. The peripheral area GA is an area between the outer perimeter of the detection area AA and the outer edges of the sensor base member 210 and is an area not provided with the photodiodes PD.

The gate line drive circuit 16 and the signal line selection circuit 17 are provided in the peripheral area GA. Specifically, the gate line drive circuit 16 is provided in an area extending along the second direction Dy in the peripheral area GA. The signal line selection circuit 17 is provided in an area extending along the first direction Dx in the peripheral area GA, and is provided between the sensor 10 and the detection circuit 48.

In the following description, the first direction Dx is one direction in a plane parallel to the sensor base member 210. The second direction Dy is one direction in the plane parallel to the sensor base member 210 and is a direction orthogonal to the first direction Dx. The second direction Dy may non-orthogonally intersect the first direction Dx. The third direction Dz is a direction orthogonal to the first direction Dx and the second direction Dy and is a direction normal to a principal surface of the sensor base member 210. The term “plan view” refers to a positional relation when viewed along a direction orthogonal to the sensor base member 210.

The light sources 53 are provided on the first light source base member 51 and are arranged along the second direction Dy. The light sources 54 are provided on the second light source base member 52 and are arranged along the second direction Dy. The first light source base member 51 and the second light source base member 52 are electrically coupled to the control circuit 122 and the power supply circuit 123 through respective terminals 124 and 125 provided on the control substrate 121.

For example, inorganic light-emitting diodes (LEDs) or organic electroluminescent (EL) diodes (organic light-emitting diodes (OLEDs)) are used as the light sources 53 and 54. The light sources 53 and 54 emit light having different wavelengths from each other.

Light emitted from the light sources 53 is reflected by the surface of the object to be detected Fg and enters the sensor 10. As a result, the sensor 10 can detect the fingerprint by detecting the shape of the asperities on the surface of the finger or the like. Light emitted from the light sources 54 is mainly reflected in the object to be detected Fg, or transmitted through the object to be detected Fg, and enters the sensor 10. As a result, the sensor 10 can detect the information on the living body in the finger or the like. Examples of the information on the living body include, but are not limited to, the pulse waves, the pulsation, and the vascular image in the finger or the palm. That is, the detection device 1 may be configured as the fingerprint detection device that detects the fingerprint or the vein detection device that detects the pattern of the blood vessels such as the veins.

The arrangement of the light sources 53 and 54 illustrated in FIG. 11 is merely an example, and can be changed as appropriate. The detection device 1 is provided with a plurality of types of the light sources 53 and 54 as light sources. However, the light sources are not limited thereto, and may be of one type. For example, the light sources 53 and 54 may be arranged on each of the first and the second light source base members 51 and 52. The light sources 53 and 54 may be provided on one light source base member, or three or more light source base members. Alternatively, only at least one light source needs to be disposed.

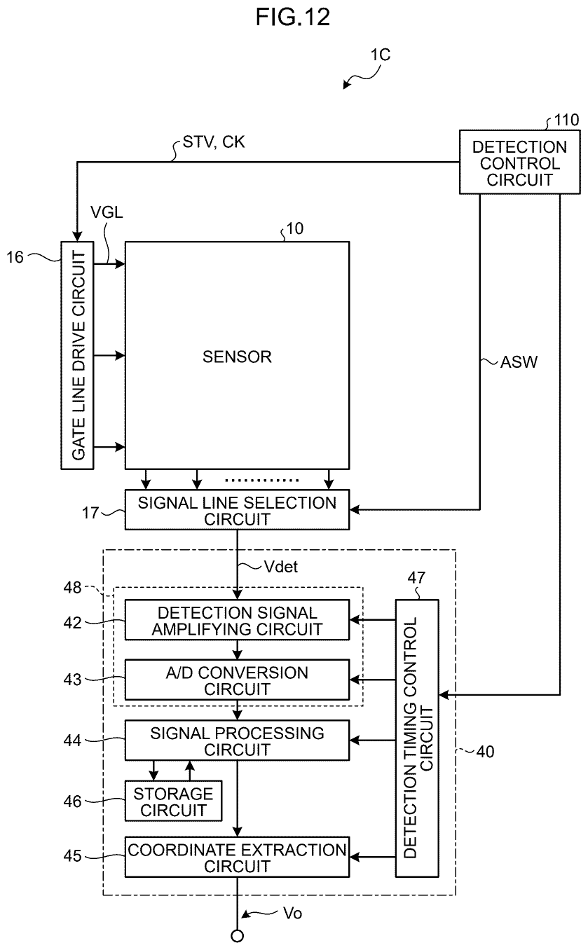

FIG. 12 is a block diagram illustrating an exemplary configuration of the detection device according to the second embodiment. As illustrated in FIG. 12, the detection device 1C further includes a detection control circuit 110 and a detector (detection signal processing circuit) 40. The control circuit 122 includes one, some, or all functions of the detection control circuit 110. The control circuit 122 also includes one, some, or all functions of the detector 40 other than those of the detection circuit 48.

The sensor 10 includes the photodiodes PD. Each of the photodiodes PD included in the sensor 10 outputs an electrical signal in response to light irradiating the photodiode PD as the detection signal Vdet to the signal line selection circuit 17. The sensor 10 performs the detection in response to a gate drive signal VGL supplied from the gate line drive circuit 16.

The detection control circuit 110 supplies respective control signals to the gate line drive circuit 16, the signal line selection circuit 17, and the detector 40 to control operations of these components. The detection control circuit 110 supplies various control signals including, for example, a start signal STV and a clock signal CK to the gate line drive circuit 16. The detection control circuit 110 also supplies various control signals including, for example, a selection signal ASW to the signal line selection circuit 17. The detection control circuit 110 also supplies various control signals to the light sources 53 and 54 to control the lighting and non-lighting of the respective light sources 53 and 54.

The gate line drive circuit 16 drives a plurality of gate lines GL (refer to FIG. 13) based on the various control signals. The gate line drive circuit 16 sequentially or simultaneously selects the gate lines GL, and supplies the gate drive signals VGL to the selected gate lines GL. Through this operation, the gate line drive circuit 16 selects the photodiodes PD coupled to the gate lines GL.

The signal line selection circuit 17 includes a switch circuit that sequentially or simultaneously selects the signal lines SL (refer to FIG. 13). The signal line selection circuit 17 is a multiplexer, for example. The signal line selection circuit 17 couples the selected signal lines SL to the detection circuit 48 based on the selection signal ASW supplied from the detection control circuit 110. Through this operation, the signal line selection circuit 17 outputs the detection signals Vdet of the photodiodes PD to the detector 40.

The detector 40 includes the detection circuit 48, a signal processing circuit 44, a coordinate extraction circuit 45, a storage circuit 46, and a detection timing control circuit 47. The detection timing control circuit 47 controls the detection circuit 48, the signal processing circuit 44, and the coordinate extraction circuit 45 to operate synchronously based on a control signal supplied from the detection control circuit 110.

The detection circuit 48 is an analog front-end (AFE) circuit, for example. The detection circuit 48 is a signal processing circuit having functions of at least a detection signal amplifying circuit 42 and an analog-to-digital (A/D) conversion circuit 43. The detection signal amplifying circuit 42 amplifies the detection signal Vdet. The A/D conversion circuit 43 converts analog signals output from the detection signal amplifying circuit 42 into digital signals.

The signal processing circuit 44 detects predetermined physical quantities received by the sensor 10 based on output signals of the detection circuit 48. The signal processing circuit 44 is a logic circuit. The signal processing circuit 44 can detect the asperities on the surface of the finger or the palm based on the signals from the detection circuit 48 when the object to be detected Fg is in contact with or in proximity to a detection surface. The signal processing circuit 44 can detect the information on the living body based on the signals from the detection circuit 48. Examples of the information on the living body include, but are not limited to, the vascular image, the pulse waves, the pulsation, and a blood oxygen level in the finger or the palm.

The storage circuit 46 temporarily stores therein signals calculated by the signal processing circuit 44. The storage circuit 46 may be, for example, a random-access memory (RAM), a register circuit, or the like.

The coordinate extraction circuit 45 obtains detected coordinates of the asperities on the surface of the object to be detected Fg such as a finger or the like when the contact or proximity of the object to be detected Fg is detected by the signal processing circuit 44. The coordinate extraction circuit 45 also obtains detected coordinates of blood vessels in the finger or the palm. The coordinate extraction circuit 45 is a logic circuit. The coordinate extraction circuit 45 combines the detection signals Vdet output from the photodiodes PD of the sensor 10 to generate two-dimensional information indicating the shape of the asperities on the surface of the finger or the like and two-dimensional information indicating the shape of the blood vessels in the finger or the palm. The coordinate extraction circuit 45 may output the detection signal Vdet as a sensor output voltage Vo instead of calculating the detected coordinates.

FIG. 13 is a circuit diagram illustrating the detection device according to the second embodiment. FIG. 13 also illustrates a circuit configuration of the detection circuit 48. As illustrated in FIG. 13, a sensor pixel PX includes the photodiode PD, a capacitive element Ca, and a drive transistor Tr. The capacitive element Ca is capacitance (sensor capacitance) generated in the photodiode PD and is equivalently coupled in parallel to the photodiode PD.

FIG. 13 illustrates two gate lines GL(m) and GL(m+1) arranged in the second direction Dy among the gate lines GL. FIG. 13 also illustrates two signal lines SL(n) and SL(n+1) arranged in the first direction Dx among the signal lines SL. The sensor pixel PX is an area surrounded by the gate lines GL and the signal lines SL.

The drive transistors Tr are provided correspondingly to the photodiodes PD. Each of the drive transistors Tr is configured as a thin-film transistor, and in this example, configured as an n-channel metal oxide semiconductor (MOS) thin-film transistor (TFT).

Each of the gate lines GL is coupled to the gates of the drive transistors Tr arranged in the first direction Dx. Each of the signal lines SL is coupled to either the sources or the drains of the drive transistors Tr arranged in the second direction Dy. The other of the source and the drain of each drive transistor Tr is coupled to the anode of the photodiode PD and the capacitive element Ca.

The cathode of the photodiode PD is supplied with the sensor power supply signal VDDSNS from the power supply circuit 123 (refer to FIG. 11). The signal line SL and the capacitive element Ca are supplied with a sensor reference voltage COM serving as an initial potential of the signal line SL and the capacitive element Ca from the power supply circuit 123 via a reset transistor TrR.

When the sensor pixel PX is irradiated with light in an exposure period, a current corresponding to the amount of the light flows through the photodiode PD. As a result, an electric charge is stored in the capacitive element Ca. When the drive transistor Tr is turned on in a readout period, a current corresponding to the electric charge stored in the capacitive element Ca flows through the signal line SL. The signal line SL is coupled to the detection circuit 48 via an output transistor TrS of the signal line selection circuit 17. Thus, the detection device 1 can detect a signal corresponding to the amount of the light irradiating the photodiode PD for each sensor pixel PX.

During the readout period, a switch SSW is turned on to couple the detection circuit 48 to the signal line SL. The detection signal amplifying circuit 42 of the detection circuit 48 converts a current or an electric charge supplied from the signal line SL into a voltage corresponding thereto. A reference potential (Vref) having a fixed potential is supplied to a non-inverting input portion (+) of the detection signal amplifying circuit 42, and the signal line SL is coupled to an inverting input portion (−) of the detection signal amplifying circuit 42. In the present embodiment, the same signal as the sensor reference voltage COM is supplied as the reference potential (Vref) voltage. The control circuit 122 (refer to FIG. 11) calculates the difference between the detection signal Vdet when light is emitted and the detection signal Vdet when light is not emitted as the sensor output voltage Vo. The detection signal amplifying circuit 42 includes a capacitive element Cb and a reset switch RSW. During a reset period, the reset switch RSW is turned on to reset the electric charge of the capacitive element Cb.

The drive transistor Tr is not limited to the n-type TFT, and may be configured as a p-type TFT. The pixel circuit of the sensor pixel PX illustrated in FIG. 3 is merely exemplary. The sensor pixel PX may include one photodiode PD provided with a plurality of transistors.

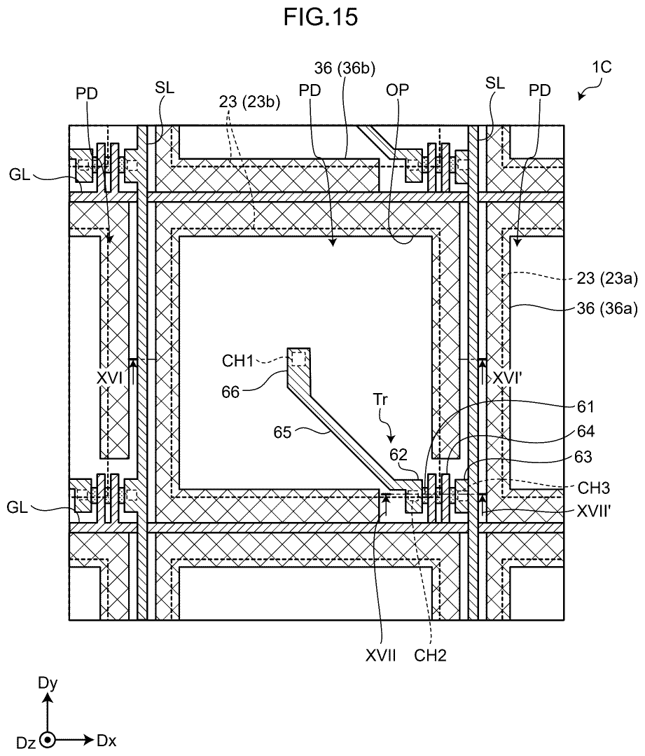

The following describes a configuration of the photodiode PD. FIG. 14 is a magnified schematic configuration view of the sensor according to the second embodiment. FIG. 15 is a plan view illustrating a light-blocking layer according to the second embodiment. FIG. 14 is a plan view illustrating a portion of the sensor 10, and is a plan view excluding a light-blocking layer 36 from FIG. 15. In FIG. 15, the light-blocking layer 36 is illustrated in a hatched manner

As illustrated in FIGS. 14 and 15, the detection device 1C includes the photodiodes PD provided on the sensor base member 210 and the light-blocking layer 36. The gate lines GL each extend in the first direction Dx, and are arranged with gaps interposed therebetween in the second direction Dy. The signal lines SL each extend in the second direction Dy and are arranged with gaps interposed therebetween in the first direction Dx. The photodiodes PD are each provided in an area surrounded by two of the gate lines GL and two of the signal lines SL, and are provided in a matrix having a row-column configuration on the sensor base member 210.

Lower electrodes 23 of the photodiodes PD are provided in a matrix having a row-column configuration on the sensor base member 210 so as to correspond to the respective photodiodes PD. As illustrated in FIG. 14, each of the lower electrodes 23 has a first side 23a and a second side 23b that intersects the first side 23a. The first sides 23a and the second sides 23b are arranged so as to be spaced from the signal lines SL and the gate lines GL, respectively.

The drive transistor Tr is provided in an area overlapping the lower electrode 23 of the photodiode PD. Specifically, the drive transistor Tr includes a semiconductor layer 61, a source electrode 62, a drain electrode 63, and a gate electrode 64. The semiconductor layer 61 extends along the gate line GL and is provided so as to intersect the gate electrode 64 in plan view. The gate electrode 64 is coupled to the gate line GL and extends in a direction (second direction Dy) orthogonal to the gate line GL.

As illustrated in FIG. 15, the light-blocking layer 36 is provided in areas overlapping the first sides 23a and the second sides 23b of the lower electrodes 23 in plan view. The light-blocking layer 36 is not provided in areas overlapping the drive transistors Tr.

In more detail, the light-blocking layer 36 includes first light-blockers 36a and second light-blockers 36b. The light-blocking layer 36 is formed in a grid pattern with the first light-blockers 36a intersecting the second light-blockers 36b. Each of the first light-blockers 36a extends in the second direction Dy. The first light-blocker 36a overlaps the first side 23a of the lower electrode 23 and extends along the first side 23a of the lower electrode 23. Each of the second light-blockers 36b extends in the first direction Dx. The second light-blocker 36b overlaps the second side 23b of the lower electrode 23 and extends along the second side 23b of the lower electrode 23.

As illustrated in FIG. 15, the opening OP is formed in an area of the light-blocking layer 36 overlapping the lower electrode 23. The opening OP of the light-blocking layer 36 is an area surrounded by two of the first light-blockers 36a and two of the second light-blockers 36b.

The shapes, the arrangement pitch, and the like of the lower electrodes 23 and the light-blocking layer 36 illustrated in FIGS. 14 and 15 are merely exemplary and can be changed as appropriate depending on the characteristics and the detection accuracy required for the detection device 1C.

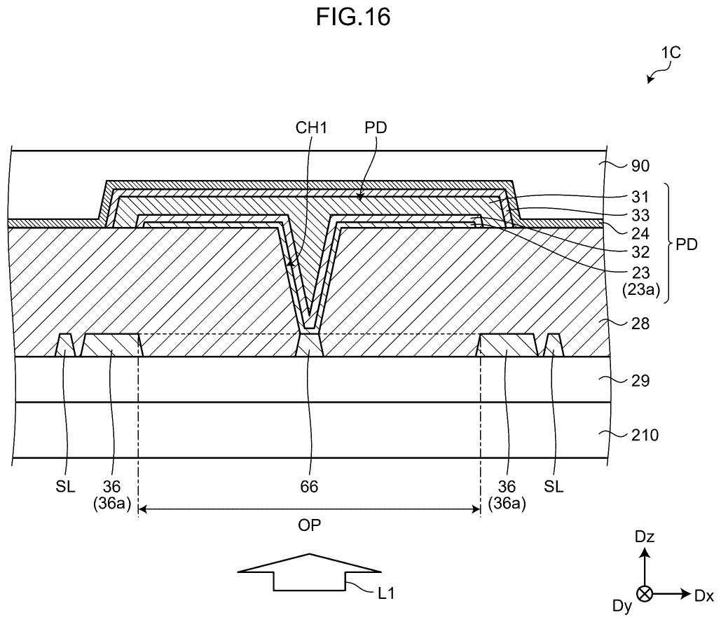

FIG. 16 is a sectional view taken along XVI-XVI′ of FIG. 15. As illustrated in FIG. 16, in the detection device 1C, a circuit forming layer 29, the light-blocking layer 36, an insulating film 28 (organic insulating film), the photodiode PD, and a sealing film 90 are stacked in this order on the sensor base member 210. The sensor base member 210 is an insulating substrate and is made using, for example, a glass substrate of quartz, alkali-free glass, or the like. The sensor base member 210 is not limited to having a flat plate shape, and may have a curved surface. In this case, the sensor base member 210 may be made of a film-like resinous material.

The circuit forming layer 29 is a layer that is provided on the sensor base member 210. The circuit forming layer 29 is provided with various transistors, such as the drive transistors Tr illustrated in FIGS. 13 and 14, and various types of wiring, such as the gate lines GL and the signal lines SL. Specifically, the circuit forming layer 29 includes the drive transistors Tr, at least part of the gate lines GL, and at least part of the signal lines SL. FIG. 16 illustrates the signal lines SL of the circuit forming layer 29 that are coupled to the drive transistors Tr. The insulating film 28 is provided on the circuit forming layer 29 including the drive transistors Tr so as to cover the signal lines SL. The insulating film 28 is an organic planarizing film formed of an organic insulating material.

The photodiode PD is provided on the insulating film 28. In more detail, the photodiode PD includes the lower electrode 23, a lower buffer layer 32, the active layer 31, an upper buffer layer 33, and an upper electrode 24. In the photodiode PD, the lower electrode 23, the lower buffer layer 32, the active layer 31, the upper buffer layer 33, and the upper electrode 24 are stacked in this order in a direction orthogonal to the sensor base member 210. The photodiode PD of the present embodiment is an organic photodiode (OPD) made using an organic semiconductor as the active layer 31.

The lower buffer layer 32, the active layer 31, the upper buffer layer 33, and the lower electrode 23 are arranged so as to be separated for each of the photodiodes PD.

The components and materials of the upper electrode 24, the upper buffer layer 33, the active layer 31, the lower buffer layer 32, and the lower electrode 23 are almost the same as those of the upper electrode 15, the upper buffer layer 14, the active layer 13, the lower buffer layer 12, and the lower electrode 11, respectively, of the detection device 1 according to the first embodiment, and are therefore not described.

The light-blocking layer 36 (first light-blocker 36a) is a metal layer or an alloy layer provided in the same layer as the signal line SL, the source electrode 62, and the drain electrode 63. The light-blocking layer 36 is provided in the areas overlapping the edge region of the lower buffer layer 32, the edge region of the active layer 31, and the edge region of the upper buffer layer 33, and an area overlapping the edge region of the lower electrode 23.

A contact hole CH1 is provided so as to penetrate the insulating film 28 in the thickness direction thereof (third direction Dz) at the central portion of the lower electrode 23. The lower electrode 23 is coupled to a coupling pad 66 at the bottom of the contact hole CH1. The lower electrode 23 is provided so as to cover the bottom of the contact hole CH1 and is conductive to the coupling pad 66 at the bottom of the contact hole CH1.

The lower buffer layer 32 and the upper buffer layer 33 are provided to facilitate holes and electrons generated in the active layer 31 to reach the lower electrode 23 or the upper electrode 24. The lower buffer layer 32 is in direct contact with the top of the lower electrode 23. The edge region of the photodiode PD is provided so as to overlap the light-blocking layer 36.

The active layer 31 is in direct contact with the top of the lower buffer layer 32. The upper buffer layer 33 is in direct contact with the top of the active layer 31, and the upper electrode 24 is in direct contact with the top of the upper buffer layer 33.

The upper electrode 24 is provided on the upper buffer layer 33. The upper electrode 24 is a cathode electrode of the photodiode PD and is continuously formed across the entire detection area AA. In other words, the upper electrode 24 is continuously provided on the photodiodes PD. The upper electrode 24 faces the lower electrodes 23 with the lower buffer layer 32, the active layer 31, and the upper buffer layer 33 interposed therebetween.

The sealing film 90 is provided on the upper electrode 24. An inorganic film, such as a silicon nitride film, or an aluminum oxide film or a resin film, such as an acrylic film, is used as the sealing film 90. The sealing film 90 is not limited to a single layer and may be a multilayered film having two or more layers obtained by combining the inorganic film with the resin film mentioned above. The sealing film 90 well seals the photodiode PD, and thus can reduce moisture entering the photodiode PD from the upper surface side thereof.

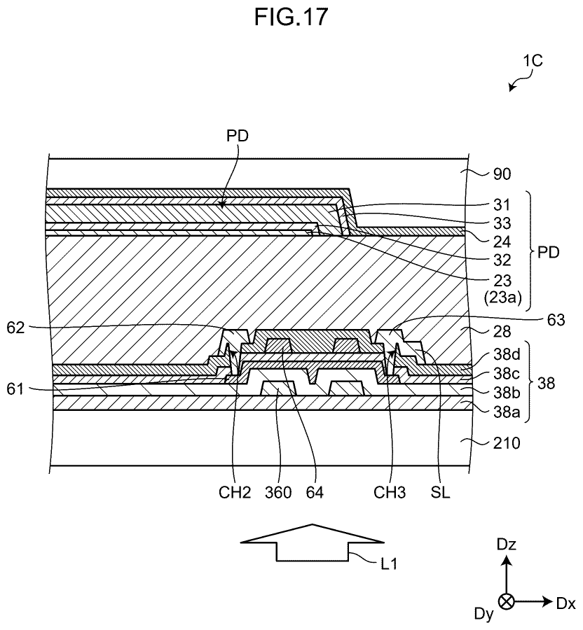

FIG. 17 is a sectional view taken along XVII-XVII′ of FIG. 15. As illustrated in FIG. 17, in the detection device 1C, an insulating film 38, a light-blocking layer 360, an insulating film 28 (organic insulating film), the photodiode PD, and the sealing film 90 are stacked in this order on the sensor base member 210.

The insulating film 38 includes a first insulating film 38a, a second insulating film 38b, a third insulating film 38c, and a fourth insulating film 38d. In the insulating film 38, a first insulating film 38a, a second insulating film 38b, a third insulating film 38c, and a fourth insulating film 38d are stacked in this order on the sensor base member 210.

The first insulating film 38a is provided on the sensor base member 210. The light-blocking layer 360 is provided on the first insulating film 38a. The light-blocking layer 360 may be conductive to the gate electrode 64, and the light-blocking layer 360 may serve as the gate electrode.

The second insulating film 38b is provided on the first insulating film 38a so as to cover the light-blocking layer 360. The semiconductor layer 61 is provided on the second insulating film 38b.

The third insulating film 38c is provided on the second insulating film 38b so as to cover the semiconductor layer 61. The gate electrode 64 is provided on the third insulating film 38c. The light-blocking layer 360 overlaps the semiconductor layer 61 and the gate electrode 64 as viewed along the third direction Dz, and is provided in a layer different from the semiconductor layer 61 and the gate electrode 64.

The fourth insulating film 38d is provided on the third insulating film 38c so as to cover the gate electrode 64. The source electrode 62 is provided on the fourth insulating film 38d on one end side of the semiconductor layer 61. The drain electrode 63 is provided on the fourth insulating film 38d on the other end side of the semiconductor layer 61. The source electrode 62 faces the drain electrode 63 in the first direction Dx with the gate electrode 64 interposed therebetween. The first insulating film 38a, the second insulating film 38b, the third insulating film 38c, and the fourth insulating film 38d are formed, for example, of a light-transmitting inorganic material such as silicon oxide or silicon nitride.

For example, polysilicon is used as the semiconductor layer 61. The semiconductor layer 61 is, however, not limited to this material, and may be a microcrystalline oxide semiconductor, an amorphous oxide semiconductor, low temperature polysilicon (LTPS), or the like.

One end side of the semiconductor layer 61 is coupled to the source electrode 62 through a contact hole CH2. The source electrode 62 is coupled to coupling wiring 65 and the coupling pad 66 and extends to a central portion of the photodiode PD (lower electrode 23). The lower electrode 23 is coupled to the coupling pad 66 through the contact hole CH1 at the central portion. Such a configuration electrically couples the source electrode 62 of the drive transistor Tr to the photodiode PD. The other end side of the semiconductor layer 61 is coupled to the drain electrode 63 through a contact hole CH3. The drain electrode 63 is coupled to the signal line SL.

The detection device 1C is configured as a bottom-illuminated optical sensor. That is, the light L1 is emitted from the light sources 53 and 54 (refer to FIG. 11) to the object to be detected Fg. The light L1 transmitted through or reflected by the object to be detected Fg is transmitted through the sensor base member 210 and is irradiated onto the lower electrode 23 of the photodiode PD. The light L1 is transmitted through the opening OP of the light-blocking layer 36 and is irradiated onto the active layer 31 of the photodiode PD. The carriers (holes and electrons) generated in the active layer 31 reach the lower electrode 23 and the upper electrode 24 through the lower buffer layer 32 and the upper buffer layer 33, respectively.

The light L1 is blocked in an area overlapping the light-blocking layer 36 and does not reach the active layer 31 located in the area overlapping the light-blocking layer 36. In more detail, the light L1 does not irradiate portions overlapping the edge region of the lower buffer layer 32, the edge region of the active layer 31, the edge region of the upper buffer layer 33, and the edge region of the lower electrode 23. This configuration reduces generation of the carriers (holes and electrons) in the portion of the active layer 31 that overlaps the light-blocking layer 36. As a result, the detection device 1 including the OPD can improve the detection accuracy.

### First Modification of Second Embodiment

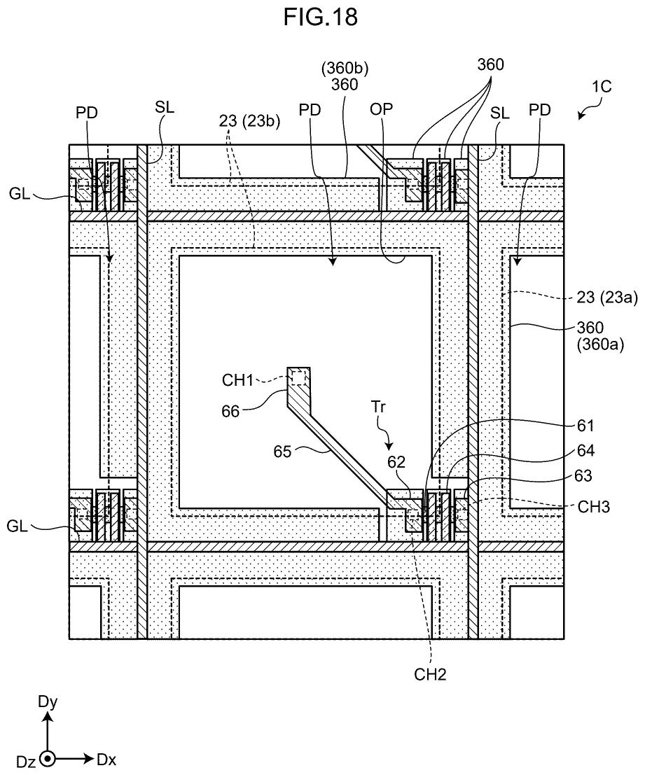

FIG. 18 is a plan view illustrating a light-blocking layer according to a first modification of the second embodiment. In the following description, the same components as those described in either of the embodiments above are denoted by the same reference numerals, and the description thereof will not be repeated.

As illustrated in FIG. 18, the light-blocking layer 360 may be provided in areas overlapping outer edge region of the lower electrode 23, the signal lines SL, and the gate lines GL, without providing the light-blocking layer 36 illustrated in FIG. 15. In this case, the light-blocking layer 360 is separately provided so as to individually overlap the source electrode 62, the drain electrode 63, and the gate electrode 64. The second light-blocker 36b is not provided in areas overlapping gaps between: the light-blocking layer 360 overlapping each of the source electrode 62, the drain electrode 63, and the gate electrode 64; a first light-blocker 360a extending along the signal line SL; and a second light-blocker 360b extending along the gate line GL.

### Second Modification of Second Embodiment

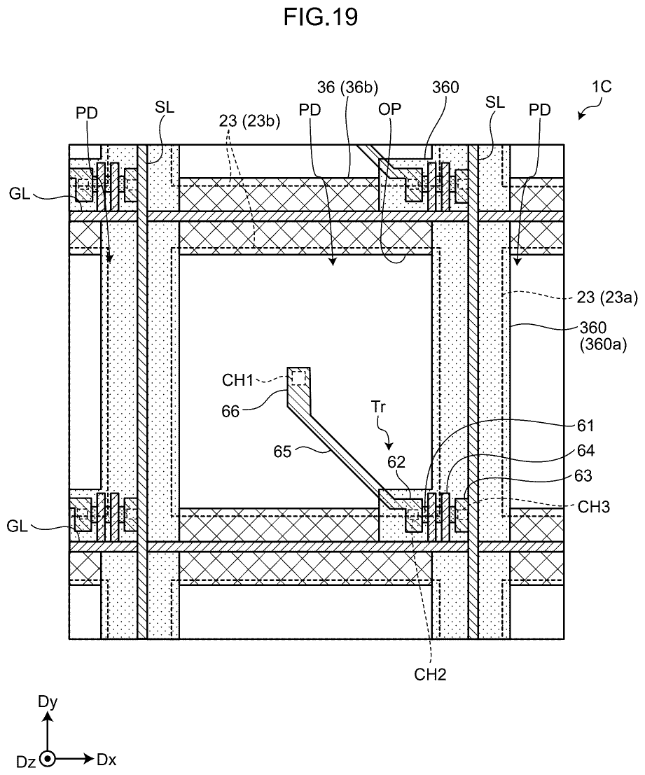

FIG. 19 is a plan view illustrating the light-blocking layer according to a second modification of the second embodiment. The light-blocking layer according the second modification includes two layers at different levels. In the following description, the same components as those described in either of the embodiments above are denoted by the same reference numerals, and the description thereof will not be repeated. As illustrated in FIG. 19, the light-blocking layer 36 illustrated in FIG. 15 and the light-blocking layer 360 illustrated in FIG. 18 may overlap each other, and the two layers may be disposed in areas overlapping the outer edge region of the lower electrode 23, the entire drive transistor Tr, the signal line SL, and the gate line GL.

In this case, in the first direction Dx, the second light-blocker 36b is provided so as to overlap the outer edge region of the lower electrode 23. The second light-blocker 36b is in the same layer as the gate line GL. In the second direction Dy, the first light-blocker 360a is provided so as to overlap the outer edge region of the lower electrode 23. The first light-blocker 360a is provided so as to overlap the entire drive transistor Tr. The first light-blocker 360a is the same layer as the signal line SL. With this configuration, the second light-blocker 36b, the first light-blocker 360a, and the entire drive transistor Tr are shielded from light without gaps.

The configuration of the photodiode PD illustrated in FIGS. 14 to 19 is merely exemplary and can be changed as appropriate. For example, the upper electrode 24 may be the anode electrode of the photodiode PD, and the lower electrode 23 may be the cathode electrode of the photodiode PD.

### Third Modification of Second Embodiment

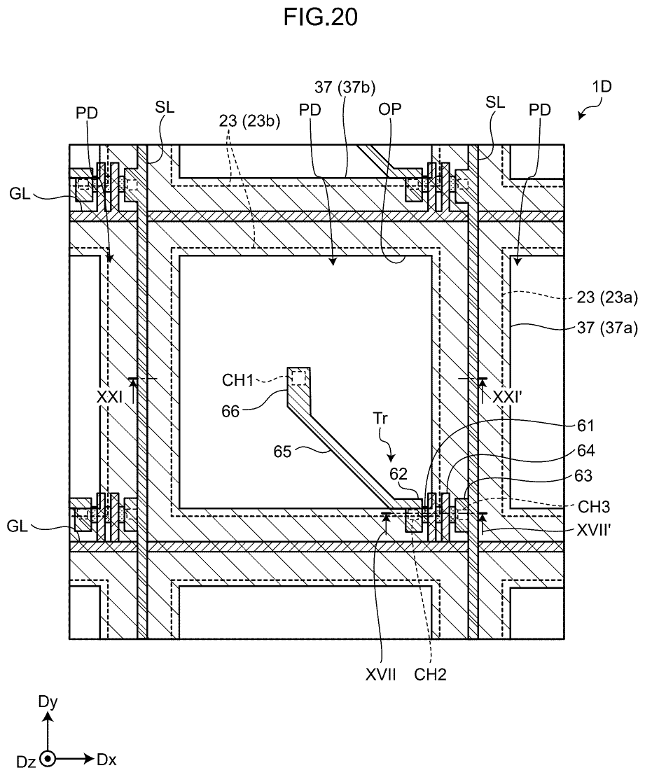

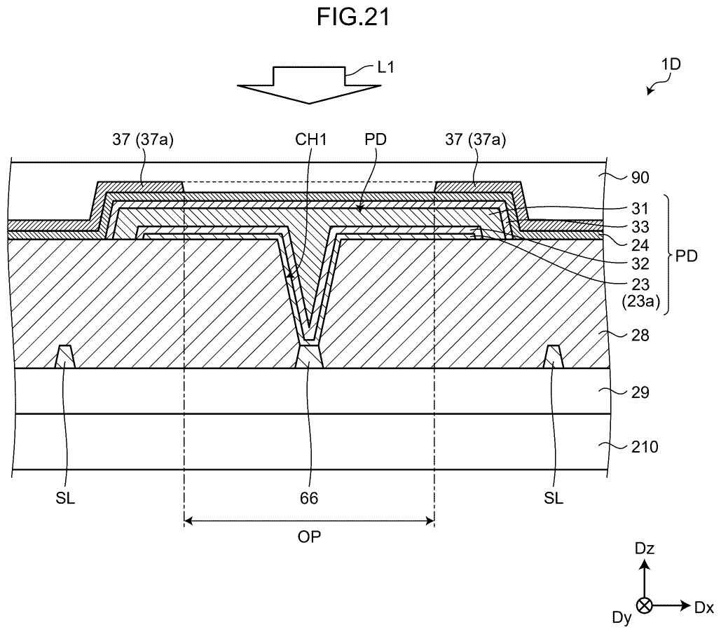

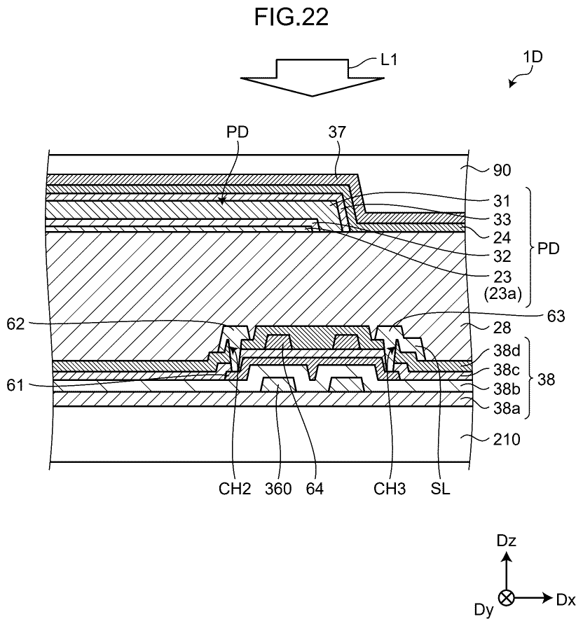

FIG. 20 is a plan view illustrating a light-blocking layer according to a third modification of the second embodiment. FIG. 21 is a sectional view taken along XXI-XXI′ of FIG. 20. FIG. 22 is a sectional view taken along XXII-XXII′ of FIG. 20. In the following description, the same components as those described in either of the embodiments above are denoted by the same reference numerals, and the description thereof will not be repeated.

As illustrated in FIG. 20, in a detection device 1D according to the third modification of the second embodiment, a light-blocking layer 37 is provided in an area overlapping the first sides 23a and the second sides 23b of the lower electrodes 23, and the drive transistor Tr in plan view. The light-blocking layer 37 is provided in areas overlapping the signal lines SL and the gate lines GL. The detection device 1D is not provided with the light-blocking layer 36 of the detection device 1C according to the second embodiment.

In more detail, the light-blocking layer 37 includes first light-blockers 37a and second light-blockers 37b. The light-blocking layer 37 is formed in a grid pattern with the first light-blockers 37a intersecting the second light-blockers 37b. Each of the first light-blockers 37a extends in the second direction Dy. The first light-blocker 37a overlaps the first side 23a of the lower electrode 23 and extends along the first side 23a of the lower electrode 23. Each of the second light-blockers 37b extends in the first direction Dx. The second light-blocker 37b overlaps the second side 23b of the lower electrode 23 and extends along the second side 23b of the lower electrode 23.

The opening OP is formed in an area of the light-blocking layer 37 overlapping the lower electrode 23. The opening OP in the light-blocking layer 37 is an area surrounded by two of the first light-blockers 37a and two of the second light-blockers 37b.

As illustrated in FIG. 21, the light-blocking layer 37 is provided on the upper electrode 24. The light-blocking layer 37 is formed of a non-light-transmitting metal layer or alloy layer. The light-blocking layer 37 is in contact with the upper electrode 24 and has the same potential as that of the upper electrode 24. In the detection device 1D, the light-blocking layer 37 is provided in the third direction Dz relative to the upper electrode 24 so as to cover a sloping surface of the upper electrode 24 covering a side surface of the active layer 31.

As illustrated in FIG. 22, in the detection device 1D, the insulating film 38, the light-blocking layer 360, the insulating film 28 (organic insulating film), the photodiode PD, the light-blocking layer 37, and the sealing film 90 are stacked in this order on the sensor base member 210.

The light-blocking layer 37 is provided in areas overlapping the whole of the semiconductor layer 61, the source electrode 62, the drain electrode 63, and the gate electrode 64.

The detection device 1D is configured as a top-illuminated optical sensor. That is, the light L1 emitted from the light sources 53 and 54 (refer to FIG. 11) and transmitted through or reflected by the object to be detected Fg is transmitted through the sealing film 90 and is irradiated onto the upper electrode 24 of the photodiode PD. The light L1 is transmitted through the opening OP in the light-blocking layer 37 and is irradiated onto the active layer 31 of the photodiode PD. The carriers (holes and electrons) generated in the active layer 31 reach the lower electrode 23 and the upper electrode 24 through the lower buffer layer 32 and the upper buffer layer 33, respectively.

Also, in the present embodiment, the light L1 is blocked in an area overlapping the light-blocking layer 37 and does not reach a portion of the active layer 31 overlapping the light-blocking layer 37. In more detail, the light L1 does not irradiate portions overlapping the edge region of the lower buffer layer 32, the edge region of the active layer 31, the edge region of the upper buffer layer 33, and the edge region of the lower electrode 23. This configuration reduces generation of the carriers (holes and electrons) in the portion of the active layer 31 that overlaps the light-blocking layer 37. As a result, the detection device 1D including the OPD can improve the detection accuracy.

The components in the embodiments described above can be combined as appropriate. Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a substrate;
a plurality of photodiodes in each of which a lower electrode, a lower buffer layer, an active layer, an upper buffer layer, and an upper electrode are stacked on the substrate in the order as listed; and
a light-blocking layer provided in an area overlapping an edge region of the lower buffer layer, an edge region of the active layer, an edge region of the upper buffer layer, and an edge region of the lower electrode in plan view, wherein
the upper electrode covers the lower buffer layer, the active layer, the upper buffer layer, and the lower electrode, and
the lower buffer layer, the active layer, the upper buffer layer, and the lower electrode are arranged so as to be separated for each of the photodiodes.

2. The detection device according to claim 1, wherein the light-blocking layer is provided between the substrate and the lower electrode in a direction orthogonal to the substrate.

3. The detection device according to claim 1, wherein the light-blocking layer is provided so as to cover an edge region of the upper electrode.

4. The detection device according to claim 1, comprising a drive transistor at a location overlapping the lower electrode, wherein
the drive transistor comprises a source electrode and a drain electrode, and
the light-blocking layer is a metal layer or an alloy layer provided in the same layer as the source electrode and the drain electrode.

5. The detection device according to claim 1, wherein
the light-blocking layer is provided so as to cover a sloping surface of the upper electrode covering a side surface of the active layer, and
the light-blocking layer is a metal material or an alloy material.

6. The detection device according to claim 2, comprising a light source configured to emit light to an object to be detected, wherein
the light emitted from the light source and transmitted through or reflected by the object to be detected is irradiated onto the lower electrode of the photodiode.

7. The detection device according to claim 4, comprising a light source configured to emit light to an object to be detected, wherein
the light emitted from the light source and transmitted through or reflected by the object to be detected is irradiated onto the lower electrode of the photodiode.

8. The detection device according to claim 3, comprising a light source configured to emit light to an object to be detected, wherein
the light emitted from the light source and transmitted through or reflected by the object to be detected is irradiated onto the upper electrode of the photodiode.

9. The detection device according to claim 5, comprising a light source configured to emit light to an object to be detected, wherein
the light emitted from the light source and transmitted through or reflected by the object to be detected is irradiated onto the upper electrode of the photodiode.
